(19) United States
(12) Patent Application Publication
(10) Pub. No.: US 2007/0050015 A1
(43) Pub. Date: Mar. 1, 2007

(54) Endoluminal prosthesis adapted to deployment in a distorted branched body lumen and method of deploying the same

(75) Inventors: Mark E. O'Brien, Natick, MA (US); John E. Sherry, Needham, MA (US)

Correspondence Address: RATNERPRESTIA, P O BOX 980, VALLEY FORGE, PA, 19482-0980, US

(73) Assignee: Scimed Life Systems, Inc.

(21) Appl. No.: 11/211,895

(22) Filed: Aug. 25, 2005

(51) Int. Cl. A61F 2/06

(52) U.S. Cl. 623/1.35

(57) ABSTRACT

An endoluminal device adapted to be deployed in a branched body lumen is provided by intraluminal delivery from an access location remote from the location at which the device is to be deployed, i.e. by introduction through one of the branches of the branched body lumen. The device comprises an unbranched portion, a first branch portion and second branch portion in communication with the unbranched portion, and a distortion element removeably connected to the first branch portion for manipulating the geometry of the second branch portion. The distortion element extends along the first branch portion and back to the access location.

### BACKGROUND OF THE INVENTION

It is known to treat an abdominal aortic aneurysm (AAA) by endoluminally implanting a bifurcated stent-graft in order to bypass the aneurysm. Stent-grafts adapted for this use include a number of designs. A large class of these designs incorporates a modular aspect that brings the benefits of reducing the size of each individual sub-component to be implanted and facilitating a customized fit for each patient. A downside of modular design is that it necessitates in situ assembly of the sub-components. A common in situ assembly process of a two-piece device includes an initial insertion of a bifurcated component whose branch portions are of differing lengths; so called “long leg-short leg devices.” The bifurcated component is inserted from a remote location into and up an ipsilateral iliac artery and positioned in the aortic neck so that the second branch portion directs luminal flow into the contralateral iliac artery. Next, a leg extension component is engaged with the second branch portion to extend the bifurcated device into the contralateral iliac artery. This can be achieved by inserting a guidewire from a remote location into the contralateral iliac artery and through the aperture of the second branch portion. The leg extension component is then delivered along the guidewire and into engagement with the second branch portion of the bifurcated component.

In patients with large saccular aneurysms, the aortic neck is typically angularly offset significantly from the iliac arteries. The offset hinders insertion of the leg extension component guidewire into the second branch portion aperture because the guidewire approaches the aperture from an acute angle.

### SUMMARY OF THE INVENTION

The present invention relates to a prosthetic device for facilitating endoluminal assembly of a modular bifurcated endoluminal device in a branched body lumen from a remote location. The invention is particularly advantageous in the treatment of large saccular AAA's with a distorted aortic neck.

Generally, an endoluminal device is adapted to be deployed in a branched body lumen, from an access location remote from the location at which the device is to be deployed, by introduction through one of the branches of the branched body lumen. The device comprises an unbranched portion and at least two branch portions in communication with said unbranched portion. The branch portions comprise a first branch portion adapted to be deployed in one branch of the body lumen and a second branch portion adapted to be directed toward a second branch of the body lumen. The device further comprises a distortion element removeably connected to the second branch portion for manipulating the geometry of the second branch portion. The distortion element extends along the first branch portion to the access location. The distortion element can be an elongated filament such as a wire, a yarn, or a cable. The removable connection can be a slipknot, a ring, or an eyelet. Optionally, the present invention also includes: a second endoluminal device adapted to be mated with the second branch portion of the first device; the methods for using and inserting those devices, and apparatus therefore.

### BRIEF DESCRIPTION OF THE DRAWINGS

The invention is best understood from the following detailed description when read in connection with the accompanying drawings. It is emphasized that, according to common practice, the various features of the drawings are not to scale. On the contrary, the dimensions of the various features may be arbitrarily expanded or reduced for clarity. Included in the drawings are the following Figures:

FIG. 1 illustrates a typical large saccular aneurysm having an offset neck;

FIG. 2A illustrates a prior art bifurcated endoluminal device having a long leg and a second branch portion;

FIG. 2B illustrates use of the prior art device depicted in FIG. 2A in a distorted aneurysm;

FIG. 3A illustrates a first device embodiment of the present invention;

FIG. 3B illustrates the device of FIG. 3A in use in a distorted aneurysm;

FIG. 3C illustrates the device of FIG. 3A in use in a distorted aneurysm showing attachment of an optional leg extension;

FIG. 4A illustrates a second device embodiment of the present invention;

FIG. 4B illustrates the device of FIG. 4A in use in a distorted aneurysm;

FIG. 4C illustrates the device of FIG. 4A in use in a distorted aneurysm showing attachment of an optional leg extension;

FIG. 5A illustrates an introducer embodiment according to the present invention showing the loaded endoluminal device fully collapsed within the introducer;

FIG. 5B illustrates the introducer of FIG. 5A with the loaded endoluminal device partially deployed;

FIG. 5C illustrates the introducer of FIG. 5A with the loaded endoluminal device deployed;

FIGS. 6A-C are cross-sectional views of the loaded introducer shown in FIG. 5A at the respective cross-section planes 6A-6A, 6B-6B, and 6C-6C;

FIG. 7 illustrates an exemplary second introducer for deployment of a leg-extending second device;

FIG. 8A illustrates an optional embodiment of the device including an everted leg in use in a distorted aneurysm; and

FIG. 8B illustrates the everted leg embodiment of FIG. 8A with the everted leg deployed.

### DETAILED DESCRIPTION OF THE INVENTION

The invention will next be illustrated with reference to the figures wherein the same numbers indicate similar elements in all figures. Such figures are intended to be illustrative rather than limiting and are included herewith to facilitate the explanation of the apparatus of the present invention.

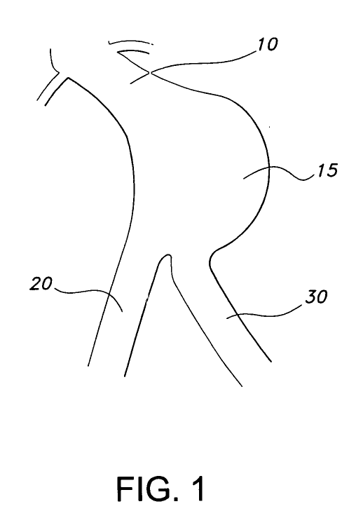

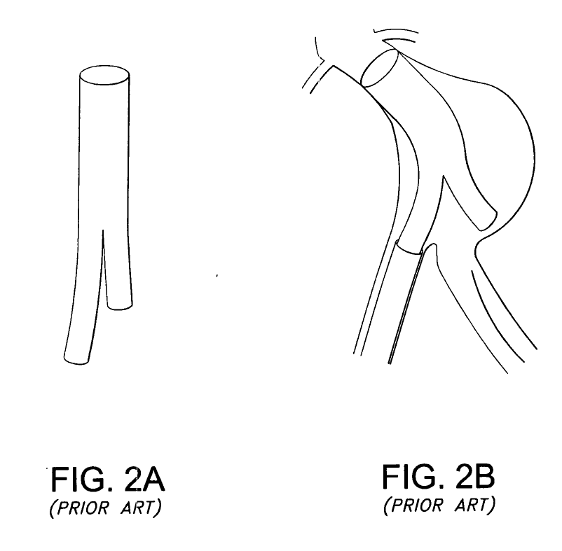

FIG. 1 illustrates an exemplary AAA having a large saccular portion 15 and a tortured neck 10 distorted from its natural vertical alignment. Ipsilateral iliac artery 20 and contralateral iliac artery 30 are also indicated. FIG. 2A depicts a bifurcated “long leg-second branch portion” prosthetic device used in the prior art to treat AAA's. FIG. 2B illustrates the difficulties in an exemplary prior art device being implanted in a large saccular AAA having a tortured neck.

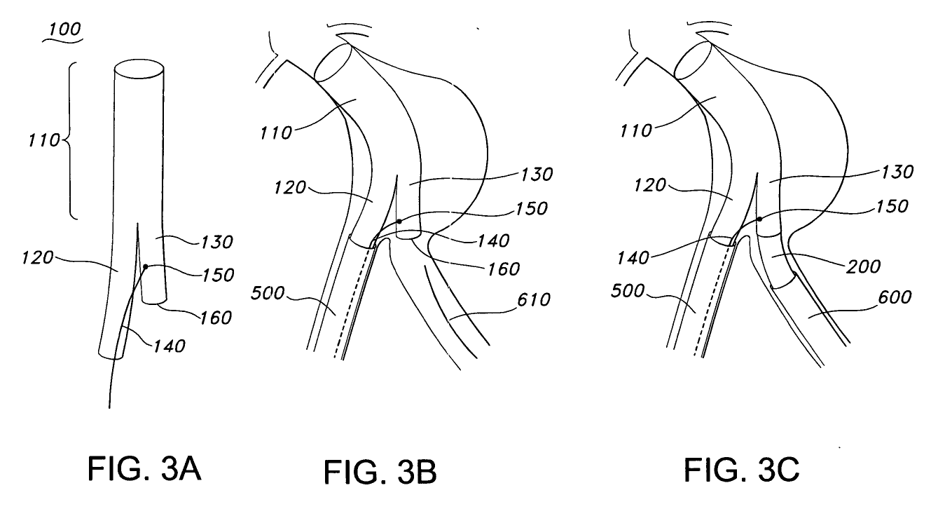

As proposed herein and as shown in FIG. 3A, an embodiment of the present invention is a bifurcated endoluminal device 100 that includes a distortion element 140 removeably connected to second branch portion 130. Distortion element 140 can take the form of any elongated filament that is capable of translating a pulling force from a remote location to the second branch portion 130. Examples of a suitable elongated filament are a wire, yarn, or cable. Distortion element 140 is connected to second branch portion 130 by a removable connection means 150 such as a slipknot, ring, or eyelet by which distortion element 140 can be detached following its use.

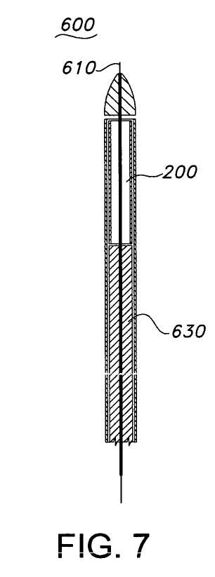

FIG. 3B shows the device in usewhereby distortion element 140 enables the surgeon to effectively pull second branch portion 130 toward first branch portion 120 by applying tension or otherwise manipulating distortion element 140. Thus, the surgeon is able to facilitate the location of open end 160 of second branch portion 130 with guidewire 610. Using guidewire 610, the surgeon can optionally deploy and mate a second device (200) to second branch portion (130) using second introducer 600 (an example of which is illustrated at FIG. 7) as needed to extend second branch portion (130) as shown in FIG. 3C. When distortion element 140 is no longer needed it can be detached from second branch portion 130 by detaching removeable connection means 150 and pulling it through the ipsiliateral iliac 30. Preferably, removal is performed through introducer 500 (discussed further below) by which device 100 was originally introduced. For example, where connection means 150 is a slipknot, simply pulling on the slipknot to untie it and then pulling distortion element 140 through introducer 500. Likewise, where connection means 150 is a slip ring, pulling one end of distortion element 140 through the slip ring and then pulling distortion element 140 through introducer 500.

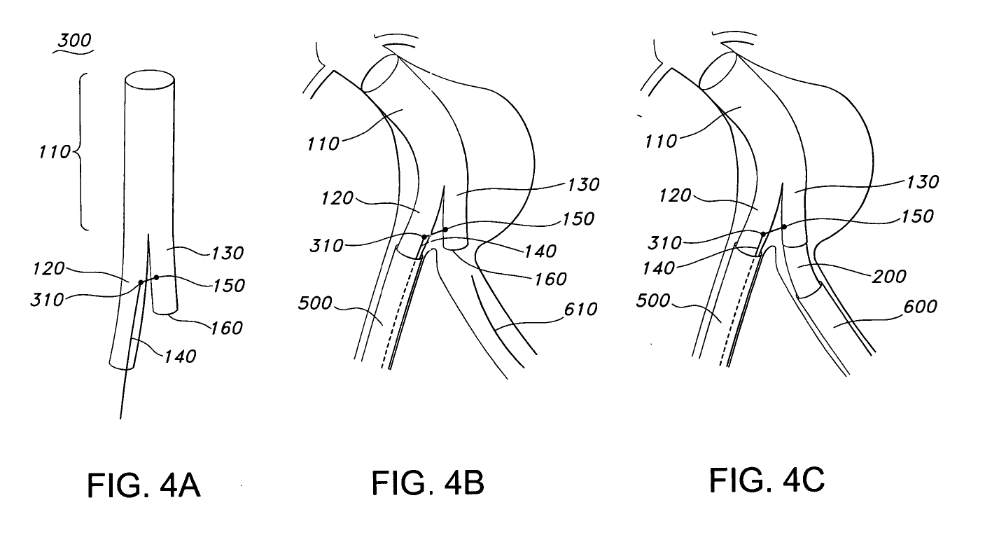

As further proposed herein and as shown in FIG. 4A, another embodiment of the present invention is a bifurcated endoluminal device 300. Bifurcated device 300 includes the features of bifurcated device 100 and further incorporates a removeable pivot point means 310 positioned on first branch portion 120 of device 300. Pivot point means 310 serves as an axis around which pulling force exerted by the surgeon can be re-directed laterally towards second branch portion 130. Pivot point means 310 may be any structure capable of performing this redirection such as a ring, hook, button, or nipple. FIG. 4B illustrates use of pivot means 310 in combination with distortion element 140 and connection means 150 to orient second branch portion 130 to facilitate insertion of guidewire 610. Using guidewire 610, the surgeon can optionally deploy and mate a second device (200) to second branch portion (200) using second introducer (600) as needed to extend second branch portion (130) as shown in FIG. 4C.

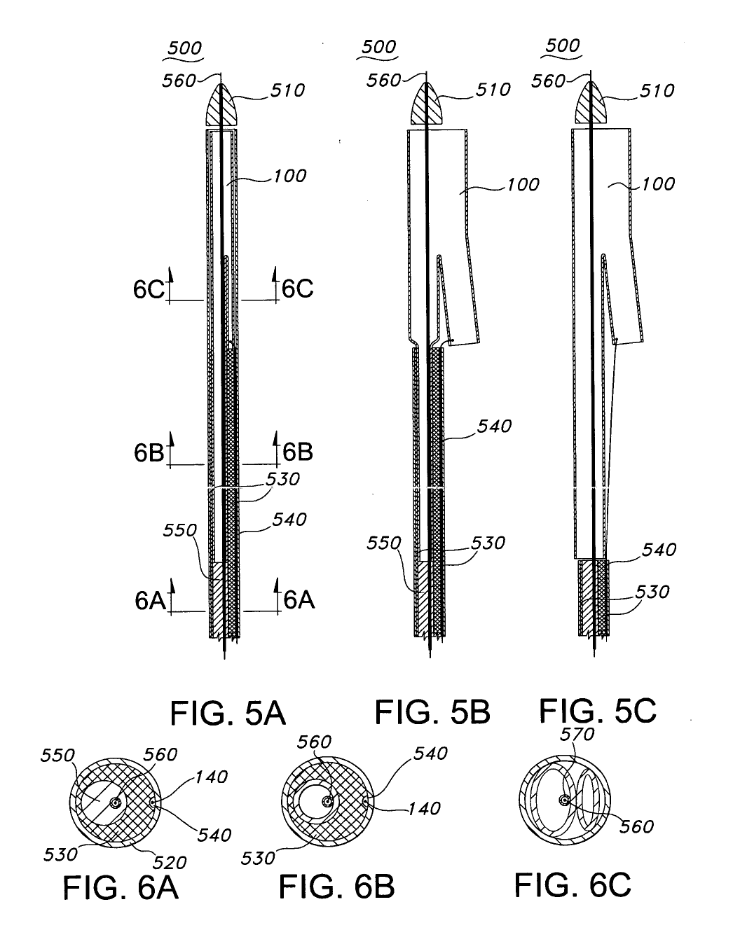

While the invention may be introduced in any number of ways which may be devised by those skilled in the art, one way of doing so is by the deployment apparatus illustrated in FIGS. 5A, 5B, 5C and 6A, 6B, and 6C.

More specifically, in FIG. 5A and in FIGS. 6A, 6B, and 6C there is shown bifurcated endoluminal device 100 compressed for endoluminal placement in an outer sheath 520 and mounted on guidewire tube 570 through which extends guidewire 560, extending from the external access point, where the delivery apparatus enters the vasculature, to a nose cone 510 just distal (with respect to the access point) of device 100. An inner sheath 530 terminates just proximal of the proximal end of second branch portion 130 and a pusher element 550 is disposed just proximal of first branch portion 10. Distortion element 140, attached to second branch portion 130 by removeable connection means 150, includes a return length or lengths extending within outer sheath 520 in a distortion element channel 540 to the external access point.

It will be noted that inner sheath 530 generally occupies the space within outer sheath 520 not otherwise occupied by pusher element 550 except for distortion element channel 540 which permits passage of distortion element 140.

FIG. 5B illustrates deployment of bifurcated endoluminal device 100 as outer sheath 520 is retracted to the point where second branch portion 130, unbranched portion 110, and a portion of first branch portion 120 are expanded, in this case by the self expansion characteristics of their elasticity or memory metal composition. At this stage, tension can be applied to distortion element 140 (or otherwise manipulating distortion element 140), thereby constraining second branch portion 130 to a limited angular separation from first branch portion 120 and thus second branch portion 130 can be better aligned with contralateral iliac 30 (as seen in FIG. 3B).

Sequentially, FIG. 5C shows the final deployment stage of bifurcated endoluminal device 100 when outer sheath 520 and inner sheath 530 are both withdrawn to the point where the remainder of first branch portion 120 is deployed. As shown in FIG. 3B, with distortion element 140 effecting some control over the angular disposition of second branch portion 130, capture of bifurcated endoluminal device 100 from contralateral iliac 30 can then be performed by passing guidewire/snag wire 610 from contralateral iliac 30 into open end 160. Thereafter, guidewire/snagwire 610 can be used, for example, to guide second introducer 600 for deploying and mating a leg-extending second device 200 to second branch portion 130.

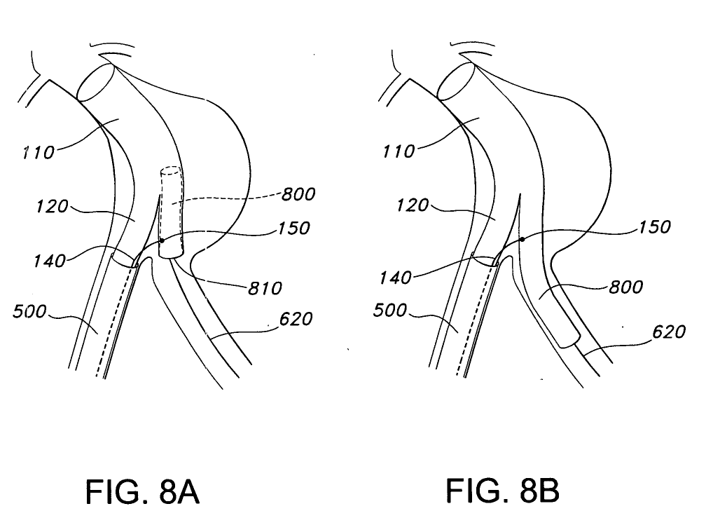

As shown in FIG. 8, an optional embodiment of bifurcated device 100 alternatively provides for the second branched portion as an everted leg (800). This optional embodiment is deployed in the same manner as previous embodiments. However, to provide an extended second branch portion the surgeon inserts snagline 620 into open end (810) of the everted leg. As above, distortion element 140 can be used to orient open end (810) to facilitate insertion of snagline 620. Using snagline 620/the surgeon can then pull everted leg 800 down and into contralateral iliac artery 30. An advantage of this embodiment has an advantage in that in many applications would required no further leg extension of the second branch portion.

Although the invention is illustrated and described herein with reference to specific embodiments, the invention is not intended to be limited to the details shown. Rather, the claims which follow are intended to be construed to encompass any modifications which may be made by those skilled in the art to which this invention pertains that nevertheless lie within the spirit and scope of this invention.

What is claimed:

1. An endoluminal device adapted to be deployed in a branched body lumen, from an access location remote from the location at which the device is to be deployed, by introduction through one of the branches of the branched body lumen, said device comprising:
(a) an unbranched portion;
(b) at least two branch portions in communication with said unbranched portion, comprising
(i) a first branch portion adapted to be deployed in one branch of the body lumen and
(ii) a second branch portion adapted to be directed toward a second branch of the body lumen; and
(c) a distortion element removably connected to said second branch portion for manipulating the geometry of the second branch portion, said distortion element extending along the first branch portion to said access location.

2. An endoluminal device, as recited in claim 1, wherein said branched body lumen is the lower aorta and the branches thereof are the iliac arteries, and the endoluminal device is a structurally supported graft.

3. An endoluminal device, as recited in claim 2, wherein the structurally supported graft is a stent graft.

4. The device of claim 1 wherein said endoluminal device is a first device and further comprising:
(d) a second device adapted to be endoluminally deployed, from a second access location in a second branch lumen, said second device also adapted to be mated with the second branch portion of the first device.

5. The device of claim 1 wherein the distortion element comprises an elongated filament.

6. The device of claim 5 wherein the elongated filament is a wire.

7. The device of claim 5 wherein the elongated filament is a yarn.

8. The device of claim 5 wherein the elongated filament is a cable.

9. The device of claim 1 wherein the removable connection means is a slipknot.

10. The device of claim 1 wherein the removable connection means is a ring.

11. The device of claim 1 wherein the removable connection means is an eyelet.

12. An endoluminal device as recited in claim 1 wherein said second branch portion comprises:
an at least partially everted portion folded within said unbranched portion and adapted to be extended into the second branch of the body lumen by unfolding said everted portion.

13. An introducer adapted to deliver the device of claim 1 in a body lumen, said introducer comprising:
(a) a tubular outer sheath having a proximal end, a distal end, and a length extending therebetween;
(b) a tubular inner sheath disposed within said outer sheath;
(c) a channel disposed between the outer sheath and the inner sheath, said channel adapted to communicate said distortion element from said second branch portion of said endoluminal device to said access location for manipulation of the second branch portion.

14. A system for deploying the device of claim 1, said system comprising:
an introducer adapted to deliver said device; and
said device loaded into said introducer.

15. A system for deploying the device of claim 4, said system comprising:
a first introducer adapted to deliver said first device;
said first device loaded into said first introducer;
a second introducer adapted to deliver said second device; and
said second device loaded into said second introducer.

16. A method for deploying the device of claim 1, from an access location in a first branch of a bifurcated body lumen from a proximal location, the method, the method comprising the steps of:
(a) deploying said device to a location where the unbranched portion is disposed in the bifurcated lumen and the first branch portion is disposed in that part of the first branch of the bifurcated lumen adjacent the bifurcation thereof and the second branch portion of the device is located in a second branch of the body lumen adjacent the first branch of the body lumen; and
(b) manipulating the distortion element to distort the second branch portion of the device.

17. The method of claim 16, further including a second device adapted to be endoluminally deployed from a second access location in a second branch lumen, said second device also adapted to be mated with the second branch portion of the first device, the method further comprising the steps of:
(c) deploying the second device in the second branch of the bifurcated lumen and mating it with the second branch of the first device.
